(19) United States
(12) Patent Application Publication
(10) Pub. No.: US 2021/0386103 A1
(43) Pub. Date: Dec. 16, 2021

(54) LETTUCE EXTRACT

(71) Applicant: FIRMENICH SA, Satigny (CH)

(72) Inventors: Xiu-Feng He, Shanghai (CN); Christian Starkenmann, Onex (CH); Christoph Cerny, Shanghai (CN); Yi-Min Wang, Shanghai (CN)

(21) Appl. No.: 17/420,517

(22) PCT Filed: Apr. 23, 2020
(86) PCT No.: PCT/EP2020/061381

§ 371 Date: Jul. 2, 2021

(30) Foreign Application Priority Data
Apr. 26, 2019 (CN) PCT/CN2019/084438

(51) Int. Cl. A23L 27/10; B01D 11/02; A23L 33/105; A23L 29/00; B01D 3/10; B01D 3/38; A23L 27/00

(52) U.S. Cl. CPC A23L 27/11; B01D 11/0288; A23L 33/105; A23L 27/88; B01D 3/10; B01D 3/38; A23L 29/045

(57) ABSTRACT

Described herein are an extract from lettuce (Lactuca sativa), in particular of stalk lettuce (Lactuca sativa var. angustana), including 2-acetyl-1-pyrroline and, optionally, precursors of 2-acetyl-1-pyrroline as well as flavoring compositions including such an extract and a method for the preparation of such an extract. Also described herein are the use of such an extract as a taste or flavor ingredient in a consumer product and a method of enhancing, improving, modifying the taste or flavor of a consumer product by making use of such an extract.

### TECHNICAL FIELD

The present invention relates to an extract from lettuce (Lactuca sativa), in particular of stalk lettuce (Lactuca sativa var. angustana), comprising 2-acetyl-1-pyrroline and, optionally, precursors of 2-acetyl-1-pyrroline as well as flavoring compositions comprising such an extract and a method for the preparation of such an extract. The invention further concerns the use of such an extract as a taste or flavor ingredient in a consumer product and a method of enhancing, improving, modifying the taste or flavor of a consumer product by making use of such an extract.

### BACKGROUND

Rice is a staple food and major food grain crop for two-thirds of the population in the world. China and India are major rice producers. Aroma is one of the most important characteristics of rice, especially when taking consumer acceptance as criterion. Nowadays consumers have become more conscious of the quality of the rice that they consume. They often prefer fragrant rices due to their characteristics and pleasant odour. Hence, demand for aromatic rice is increasing in both domestic and international markets. Thus, rice aroma has gained a leading importance as a quality character. Hence, basmati rice from India and Pakistan, and jasmine rice from Thailand fetch a higher market price.

2-acetyl-1-pyrroline (2-AP) was identified as the principal aroma compound responsible for the pleasant aroma in scented rice. Moreover, 2-AP is responsible for the “popcorn aroma” in food products, and has a very low odour threshold (0.1 μg/kg). Therefore, it can still be detected by the human nose at very low concentrations.

2-AP was identified for the first time as the most important flavour compound in cooked rice. However, since its discovery, 2-AP has continued to reveal its presence in a large variety of living systems and food products. Apart from living systems and food products, 2-AP can be formed in Maillard reactions in low yield. The amino acids proline and ornithine are discussed in literature as precursors of 2-AP by reacting to 2-AP upon thermal processing.

The pyrroline ring of 2-AP makes the compound highly unstable. As far as commercial use is concerned, the unstable nature of 2-AP makes it unsuitable for commercial synthesis. Moreover, natural or also called “clean label” versions are clearly preferred over synthetic ones. Therefore, other ways need to be identified to provide foods with the desired 2-AP aroma.

### DESCRIPTION OF THE FIGURES

FIG. 1—Mass spectrum of 2-AP.

FIG. 2—Mass spectrum of O-(2,3,4,5,6-Pentafluorobenzyl) hydroxylamine (PFBHA) derivative of 2-AP.

FIG. 3—Calibration curve of 2-AP using pandan leave essence (10 fold).

FIG. 4—Amount of 2-AP in function of temperature applied during a thermal process of the lettuce core stem.

FIG. 5—Amount of 2-AP in function of incubation duration at 100° C.

FIG. 6—solid phase micro extraction and gas chromatography coupled with a mass spectrometer (SPME-GC/MS) analysis for 2-AP among 7 different batches of lettuce (by different parts) before and after incubation at 1001 for 50 min. Peak areas are shown for extracted ion m/z 83.

FIG. 7—Stability of 2-AP in the basic fraction of the lettuce essence.

FIG. 8—Concentration of 2-AP by solid-phase extraction (SPE).

FIG. 9—Level of 2-AP in cooked lettuce supernatant and residue.

FIG. 10—Level of 2-AP before and after incubation for both normal supernatant and reconstituted supernatant after lyophilization.

FIG. 11—SPME-GC/MS analysis (EIC of m/z 83) of plain rice (upper line) and rice cooked (lower line).

### DETAILED DESCRIPTION

The present invention relates to an extract from lettuce (Lactuca sativa) comprising 2-acetyl-1 -pyrroline (2-AP) and, optionally, precursors of 2-acetyl-1 -pyrroline.

An extract is a preparation, wherein the desired compounds are comprised in concentrated form compared to the food matrix of which they were extracted from.

According to any embodiment, the extract from lettuce (Lactuca sativa) is an extract from stalk lettuce (Lactuca sativa var. angustana), which is also called Chinese lettuce or Stem lettuce.

Preferably, the stalk lettuce is a cultivar with leaf blade undulated. Even more preferably, the stalk lettuce is a cultivar with leaf blade undulated and leaf apex round. Even more preferably, the stalk lettuce is a cultivar with leaf blade undulated, leaf apex round and long and loose internode.

The extract from lettuce (Lactuca sativa), preferably stalk lettuce (Lactuca sativa var. angustana), optionally comprises precursors of 2-AP. Thereby it is understood, that the extract can comprise precursors of 2-AP, but does not necessarily have to comprise them.

Precursors of 2-AP are chemical compounds that can be converted to 2-AP via chemical reactions. The reaction of said precursors to 2-AP may be triggered by thermal processes, in particular. But also UV-light, or the presence of any kind of catalysts may trigger the formation of 2-AP from its precursors. Catalysts may be e.g. acids, bases, enzymes, or other organic/inorganic compounds as well as mixtures thereof.

Precursors of 2-AP react to 2-AP upon thermal processing at temperatures higher than room temperature, preferably between 30° C. and 100° C. Room temperature is defined as a temperature between 20° C. and 25° C. A common analy tical technique for the relative and absolute quantitative determination of 2-AP is SPME-GC/MS (solid phase micro extraction and gas chromatography coupled with a mass spectrometer).

In a preferred embodiment, precursors of 2-AP are water-soluble. A compound is considered water-soluble, if more than 1 mmol of these compounds can be dissolved in 1 liter of water at 20° C.

In a preferred embodiment, precursors of 2-AP are non-volatile. Non-volatile means that the precursors are not volatile at room temperature, i.e. they do not show a high vapor pressure at ordinary room temperature. Hence, non-volatile precursors themselves do not qualify as aroma compounds.

In a preferred embodiment, the extract is obtained from the leaves, peels, roots or the core stem of the lettuce or any mixture thereof, more preferred from leaves, peels or the core stem of the lettuce or any mixture thereof, even more preferred from peels or the core stem of the lettuce or any mixture thereof, and most preferred from the core stem of the lettuce. In a preferred embodiment, the leaves, peels, roots or the core stem of the lettuce or any mixture thereof can be used as is or can be pre-prepared, such as ground before the extraction process.

In a preferred embodiment, the extract is obtained from thermally processed lettuce (Lactuca sativa), preferably stalk lettuce (Lactuca sativa var. angustana), or parts of it. Thermal processing can occur at a temperature selected from the range of 25° C. to 100° C. preferably of 45° C. to 100° C, more preferably of 70° C. to 100° C., most preferably the temperature is comprised between 80° C. and 90° C. The thermal processing can last from a period of time selected from the range of 1 to 50 min, preferably of 30 to 50 min, more preferably of 40 to 50 min.

In a preferred embodiment, the extract is an aqueous extract or a powdered extract, more preferred a powdered extract. Aqueous extract means that the entirety of extracted material is present in solution, wherein the solvent comprises water. Powdered extract means that the entirety of extracted material is present in solid, powdered form.

In a preferred embodiment, the extract also comprises benzaldehyde, octanal, 2-acetylpyrrole and/or nonanal, or any combinations thereof. In a more preferred embodiment, the extract also comprises benzaldehyde, octanal, 2-acetylpyrrole and nonanal. High amounts of said additional compounds can be observed in an extract obtained from thermally processed lettuce. Said compounds or combination of compounds may contribute to an even more favorable flavor impression of the extract.

The invention's extract may be used as a flavoring ingredient.

The present invention also relates to the use of an extract from lettuce (Lactuca sativa), preferably stalk lettuce (Lactuca sativa var. angustana), comprising 2-acetyl-1-pyrroline and, optionally, precursors of 2-acetyl-1-pyrroline as a flavoring ingredient. In other words, it concerns a method or a process to confer, enhance, improve or modify the taste properties of a flavoring composition or of a flavored article, wherein the method comprises adding to said composition or article an effective amount of the invention's extract, e.g. to impart its typical note.

Typical effective amounts are in the order of 0.001 ppm to 1000 ppm, more preferably 0.1 ppm to 500 ppm, more preferably 0.5 ppm to 350 ppm, most preferably 1 ppm to 100 ppm, of the invention's extract based on the weight of the composition or of the article into which it is incorporated.

By “use of an extract” it has to be understood here also the use of any composition containing invention's extract which can be advantageously employed in the flavor industry.

By “taste”, it meant to designate the taste perception and the taste sensation.

Said compositions, which in fact can be advantageously employed as flavoring ingredients, are also an object of the present invention.

Therefore, the present invention also relates to a flavoring composition comprising:
i. at least an extract from lettuce (Lactuca sativa), preferably stalk lettuce (Lactuca sativa var. angustana), comprising 2-acetyl-1-pyrroline and, optionally, precursors of 2-acetyl-1-pyrroline, as defined above;
ii. at least one ingredient selected from the group consisting of a flavor carrier, a is flavoring co-ingredient and a mixture thereof; and
iii. optionally at least one flavor adjuvant.

By “flavor carrier”, it is meant a material which is substantially neutral from a flavor point of view, insofar as it does not significantly alter the organoleptic properties of flavoring ingredients. The carrier may be a liquid or a solid.

Suitable liquid carriers include, for instance, an emulsifying system, i.e. a solvent and a surfactant system, or a solvent commonly used in flavors. A detailed description of the nature and type of solvents commonly used in flavor cannot be exhaustive. Suitable solvents include, for instance, propylene glycol, triacetine, caprylic/capric triglyceride (neobee®), triethyl citrate, benzylic alcohol, ethanol, vegetable oils such as Linseed oil, sunflower oil or coconut oil or terpenes.

Suitable solid carriers include, for instance, absorbing gums or polymers, or even encapsulating materials. Examples of such materials may comprise wall-forming and plasticizing materials, such as mono, di- or trisaccharides, natural or modified starches, hydrocolloids, cellulose derivatives, polyvinyl acetates, polyvinylalcohols, proteins or pectins, or yet the materials cited in reference texts such as H. Scherz, Hydrokolloid Stabilisatoren, Dickungs- and Geliermittel in Lebensmitteln, Band 2 der Schriftenreihe Lebensmittelchemie, Lebensmittelqualität, Behr's VerlagGmbH & Co., Hamburg, 1996. Encapsulation is a well-known process to a person skilled in the art, and may be performed, for instance, using techniques such as spray-drying, agglomeration, extrusion, coacervation and the like.

By “flavoring co-ingredient” it is meant here a compound, which is used in flavoring preparations or compositions to impart a hedonic effect. In other words such an ingredient, to be considered as being a flavoring one, must be recognized by a person skilled in the art as being able to impart or modify in a positive or pleasant way the taste of a composition, and not just as having a taste.

The nature and type of the flavoring co-ingredients present in the flavoring composition do not warrant a more detailed description here, the skilled person being able to select them on the basis of its general knowledge and according to intended use or application and the desired organoleptic effect. In general terms, these flavoring co-ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, New Jersey, USA, or its more recent versions, or in other works of a similar nature, as well as in the abundant patent literature in the field of flavor. It is also understood that said co-ingredients may also be compounds known to release in a controlled manner various types of flavoring compounds.

By “flavor adjuvant” we mean here an ingredient capable of imparting additional added benefit such as a color, a particular light resistance, chemical stability, and so on. A detailed description of the nature and type of adjuvant commonly used in flavoring compositions cannot be exhaustive. Nevertheless, such adjuvants are well known to a person skilled in the art who will be able to select them on the basis of its general knowledge and according to intended use or application.

A composition consisting of at least one invention's extract and at least one flavor carrier represents a particular embodiment of the invention as well as a flavoring composition comprising at least one invention's extract, at least one flavor carrier, at least one flavor co-ingredient, and optionally at least one flavor adjuvant.

Furthermore, the invention's extract can be advantageously used in all the fields of flavor to positively impart or modify the taste of a consumer product into which said extract is added. Consequently, the present invention relates to a flavored consumer product comprising the invention's composition as defined above.

The invention's extract can be added to a flavored consumer product can be added as such or as part of an invention's flavoring composition.

For the sake of clarity, by “flavored consumer product” it is meant to designate a edible product which may be food or beverage and which can be fried or not, as well as frozen or not, low fat or not, marinated, battered, chilled, dehydrated, instant, canned, reconstituted, retorted or preserved. Therefore, a flavored article according to the invention comprises the invention's extract, as well as optional benefit agents, corresponding to taste and flavor profile of the desired edible product, e.g. a savory cube.

The nature and type of the constituents of the foodstuffs or beverages do not warrant a more detailed description here, the skilled person being able to select them on the basis of his general knowledge and according to the nature of said product.

Typical examples of said flavored consumer product include:
seasoning or condiment, such as a stock, a savory cube, a powder mix, a flavored oil, a sauce (e.g. a relish, a barbecue sauce, a dressing, a gravy or a sweet and/or a sour sauce), a salad dressing or a mayonnaise;
meat-based product, such as a poultry, beef or pork based product, a seafood, surimi, or a fish sausage;
soup, such as a clear soup, a cream soup, a chicken or beef soup or a tomato or asparagus soup;
carbohydrate-based product, such as instant noodles, rice, pasta, potatoes flakes or fried, noodles, pizza, tortillas, wraps;
dairy or fat product, such as a spread, a cheese, or regular or low fat margarine, a butter/margarine blend, a butter, a peanut butter, a shortening, a processed or flavored cheese;
savory product, such as a snack, a biscuit (e.g. chips or crisps) or an egg product, a potato/tortilla chip, a microwave popcorn, nuts, a bretzel, a rice cake, a rice cracker, etc;
confectionery, such as bakers' confectionary (e.g. a sweet pastrie or a cake), sugar confectionary (e.g. a sweet, a candy, a candied nut, a chocolate, a chewing gum and bubblegum, a sweetmeat, a pastillage, a sugarless confectionary) or chocolate confection;
oral care product, such as a toothpaste, a mouth wash, a dental care product (e.g. denture adhesive), a dental rinsing, a mouth spray, a dental powder, a dental gel or dental floss;
imitation products, such as a dairy (e.g a reformed cheese made from oils, fats and thickeners) or seafood or meat (e.g. a vegetarian meat replacer, a veggie burger) or analogues;
pet or animal food; or
beverage such as a hot drink (e.g. a tea), a soft drink including carbonated, an alcoholic drink, a ready-to-drink or a powder soft.

Some of the above-mentioned flavored consumer products may represent an aggressive medium for the invention's extract, so that it may be necessary to protect the latter from premature decomposition, for example by encapsulation.

In a preferred embodiment, the extract is added to the food product before the food product is thermally processed, i.e. before e.g. cooking, roasting, or grilling. When the extract is added to the food product before the food product is thermally processed, the amount of 2-AP increases upon thermal processing of the food product comprising the extract, as precursors of 2-AP react to 2-AP during thermal treatment.

The proportions in which the invention's extract or composition can be incorporated into the various of the aforementioned products vary within a wide range of values. These values are dependent on the nature of the consumer product to be flavored and on the desired organoleptic effect as well as the nature of the co-ingredients in a given base when the composition according to the invention are mixed with perfuming or flavoring ingredients, solvents or additives commonly used in the art.

For example, in the case of flavored consumer product, typical concentrations are in the order of 0.001 ppm to 1000 ppm, more preferably 0.1 ppm to 500 ppm, even more preferably 0.5 ppm to 350 ppm, most preferably 1 ppm to 100 ppm, of the invention's extract or composition based on the weight of the consumer product into which they are incorporated.

The present invention also relates to a method for the preparation of an extract of lettuce (Lactuca sativa), preferably stalk lettuce (Lactuca sativa var. angustana), comprising 2-acetyl-1-pyrroline and, optionally, precursors of 2-acetyl-1-pyrroline, comprising the following steps:
a) providing lettuce (Lactuca sativa), preferably stalk lettuce (Lactuca sativa var. angustana),
b) isolating therefrom an extract comprising 2-acetyl-1-pyrroline and, optionally, precursors of 2-acetyl-1-pyrroline.

The lettuce (Lactuca sativa), preferably stalk lettuce (Lactuca sativa var. angustana) can be as defined hereinabove. The lettuce, in particular the leaves, peels, roots or the core stem of the lettuce or any mixture thereof as defined above, can be provided as is or can be pre-prepared, such as grinding the lettuce before the isolation step.

The extract can be isolated by any extraction technique known in the art, such as solvent extraction or distillation, or a combination of extraction techniques.

Under solvent extraction is to be understood the extraction of the desired compounds from the food matrix by means of a solvent, such as water or any other suitable solvent or combination of solvents. Preferably, the solvent for extraction is an aqueous solvent, more preferably water.

Distillation defines a process of separating certain components or substances from a liquid mixture by using selective boiling and condensation. Hence, distillation makes use of the differences in the volatility of the components in the mixture. Different distillation techniques are well known to a person skilled in the art. Hydrodistillation is particularly suitable for the distillation of thermolabile aroma compounds. During hydrodistillation, the aroma compound in the mixture is carried away with water vapor and thus, a gentle separation of the aroma compound from the mixture is possible. Vacuum distillation is also particularly suitable for the distillation of thermolabile aroma compounds. During vacuum distillation, the aroma compound in the mixture is carried away by reduced pressure, i.e. sub-atmospheric pressure, and thus, a gentle separation of the aroma compound from the mixture in a highly efficient manner is possible.

Preferably, the extract is isolated by solvent extraction. Even more preferably, the extract is isolated by solvent extraction and subsequently distillation, such as hydrodistillation or vacuum distillation, preferably vacuum distillation.

According to a particular embodiment, method step b.) additionally comprises the following steps:
b.1.) thermally processing the lettuce to obtain a processed lettuce,
b.2.) subjecting the processed lettuce of step b.1. to hydrodistillation or vacuum distillation, preferably vacuum distillation, and
b.3.) obtaining the extract as the distillate.

Thermally processing the lettuce means that the lettuce is subjected to a heating step. Preferably, step b.1.) occurs at a temperature selected from the range of 25° C. to 100° C., more preferably of 45° C. to 100° C., even more preferably of 70° C. to 100° C., most preferably the temperature is comprised between 80° C. and 90° C. Preferably, step b.1.) lasts for a period of time selected from the range of 1 to 50 min, more preferably of 30 to 50 min.

In a preferred embodiment, the method comprises the following additional steps: b.4.) subjecting the extract obtained in step b.3. to acid/base extraction, optionally, b.5.) subjecting the extract obtained in step b.4.) to a further concentration step, preferably via solid-phase extraction, forward osmosis or pervaporation, more preferably via solid-phase extraction.

An acid/base extraction is a type of liquid-liquid extraction. It typically involves different solubility levels in water and in an organic solvent. The organic solvent may be any carbon-based liquid that is insoluble in water (non-polar solvents); common non-polar solvents are ether, ethyl acetate, dichloromethane, or pentane. Acid/base extraction may be applied to remove green aroma notes from the extract. Thereby, the extract can be acidified with an acid, such as sulfuric acid, which renders compounds, such as 2-AP, ionic and therefore soluble in the water phase. Therefore, neutral molecules, such as many green aroma notes, can be effectively extracted with a non-polar solvent, wherein ionic compounds, such as 2-AP, remain in the acidified water phase. After the extraction, the water phase can be neutralized again with a base, such as sodium carbonate.

Solid-phase extraction (SPE) is a sample preparation technique by which compounds that are dissolved or suspended in a liquid mixture are separated from other compounds in the mixture according to their physical and chemical properties. The compounds to be concentrated are absorbed on a solid-phase, whereas other compounds in the sample are not absorbed on the solid-phase. Preferably, the solid-phase is octadecyl-carbon-chain-bonded silica (C18-phase). Thus, certain compounds in the sample can be separated from each other. The compounds to be concentrated can then again be eluted from the solid-phase with a suitable solvent (eluent). Preferably, the eluent is a solution of 70% ethanol (water/ethanol; 30/70; v/v).

In another particular embodiment, method step b.) comprises the following steps:
b.1.) dissolving the lettuce in an aqueous liquid to obtain a lettuce residue and a supernatant;
b.2.) obtaining the extract as the supernatant.

The aqueous liquid may be any kind of solvent that comprises water. Preferably, the aqueous liquid is water. Preferably, the separation of the lettuce residue and the supernatant in step b.2.) is facilitated by centrifuging the lettuce residue and the supernatant to obtain a clear extract as the supernatant. Preferably, the separation of the lettuce residue and the supernatant in step b.2.) may be performed by centrifugation the lettuce residue and the supernatant following by filtration of supernatant using membrane filtration such as filtration, microfiltration, ultrafitration, forward osmosis, reverse osmosis or combination thereof.

In a preferred embodiment, the method comprises the additional step:
c1) drying the extract, preferably drying the extract by means of spray drying or lyophilization; or
c2) dilute the extract.

Drying the extract means that essentially no solvent is left in the extract after the drying step, preferably no solvent is left in the extract after the drying step. Therefore, the extract is in solid, powdered from after the drying step. Spray drying is a method of producing a dry powder from a liquid or slurry by rapidly drying with a hot gas. By contrast, freeze-drying, also known as lyophilization, is a low temperature dehydration process, which involves freezing the product, lowering pressure, then removing the ice by sublimation. Both drying methods mentioned are gentle drying methods and thus, particularly suitable for thermolabile compounds, such as many aroma compounds.

The extract according to the present invention is preferably obtainable according to the method for the preparation as defined hereinabove.

### EXAMPLES

### Example 1: Sample Preparation

As a sample, different parts of lettuce (heart stem, leaves, roots, and the milk secreted upon wounding of the peel, and the peels (milk included)) of stalk lettuce (Lactuca sativa var. angustana) are used.

All fresh lettuce samples were grounded into fine powder with a cryogenic miller cooled by liquid nitrogen to ensure homogeneity. Powdered samples were preserved at −80° C. in a freezer.

### Example 2: Analytical Methods for 2-AP

### a) SPME-GC/MS

1-2 g of sample from Example 1 is put in a 20 mL headspace vial for sampling. A Supelco DVB/CAR/PDMS SPME fiber (1 cm, film thickness 50/30 μm, Supelco, Bellefonte, Pa.) was used for SPME sampling. Prior to sampling, each sample was equilibrated at 40° C. for 10 min. After equilibration, the SPME fiber was exposed in the vial headspace for 15 min at the same temperature (40° C.). The whole sampling procedure was accomplished by a Gerstel autosampler for SPME. The fiber was then introduced into a GC/MS injection port at 250° C. for a 3 min desorption.

The GC/MS system was equipped with an Agilent 6890N GC, an Agilent 5975 mass spectrometer, and a 30 m×0.25 mm id×0.25 μm DB-1ms (J&W 122-0132) column.

### GC/MS method

Oven: 50° C. (3 min hold) to 250 C. at 100° C./min, then held 3 min. Inlet temperature: 250 C. Carrier gas: Helium. Flow rate: 0.7 mL/min. Split ratio: 25:1. Mass spectrometer ionization voltage: 70 eV. Scan range: m/z 29 to 450. Quadrupole temperature: 150° C. Ion source temperature: 230° C.

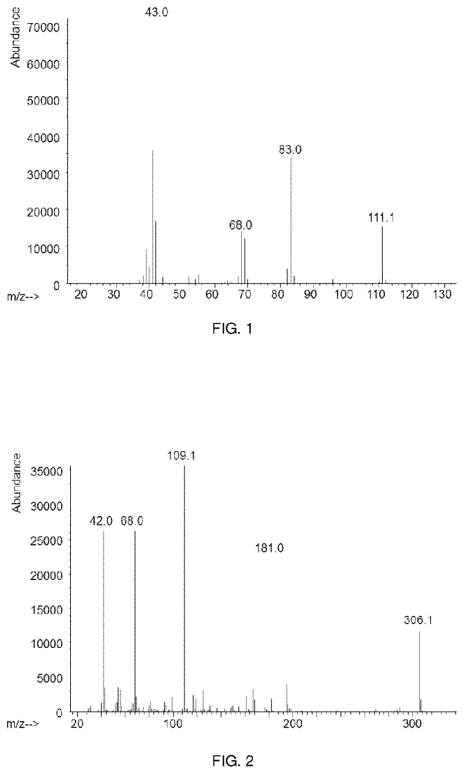

2-AP is detected with LRI of 886 on DB-1ms column and with mass spectrum as shown in FIG. 1.

### b) Derivatization and quantification of 2-AP with PFBHA

To an aqueous sample containing 2-AP excessive amount of PFBHA (O-(2,3,4,5,6-Pentafluorobenzyl) hydroxylamine) is added and mixed adequately. The reaction is allowed to stand at room temperature for 2 h, after which the reaction is extracted with ethyl acetate (EtOAc) and the organic phase is injected in GC/MS. The Linear Retention Index (LRI) value of the PFBHA derivative of 2-AP in a DB-1ms column is 1625 and its mass spectrum is shown in FIG. 2.

The GC/MS system was equipped with an Agilent 6890N GC, an Agilent 5975 mass spectrometer, and a 30 m×0.25 mm id×0.25 μm DB-1 ms (J&W 122-0132) column.

### GC/MS Method

Oven: 50° C. (5 min hold) to 300° C. at 5° C./min, then to 340° C. at 50° C./min (3 min hold).

Inlet temperature: 250° C. Carrier gas: Helium. Flow rate: 0.7 mL/min. Split ratio: 25:1. Mass spectrometer ionization voltage: 70 eV. Scan range: m/z 29 to 450. Quadrupole temperature: 150° C. Ion source temperature: 230° C.

For lettuce sample with insoluble components, the sample is first processed with ultrasonic at 45° C. for 30 min and then filtered. A n excessive amount of PFBHA is added to the filtrate and mixed adequately. The reaction is allowed to stand at room temperature for 2 h, after which the reaction is extracted with ethyl acetate (EtOAc) and the organic phase is injected in GC/MS.

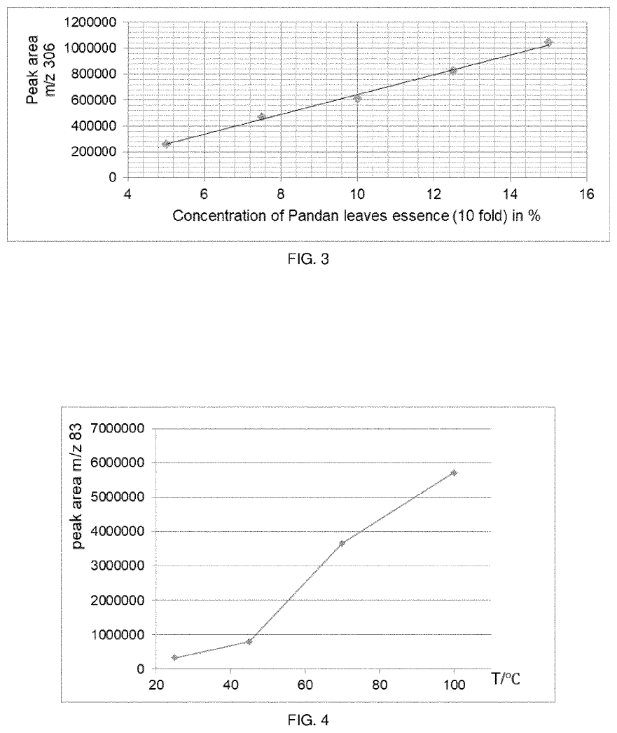

Calibration curve of 2-AP using this method was established with Pandan leave essence 10 fold (FIG. 3).

c) Quantification of 2-AP with internal standards

Quantification is carried out with vanillin as internal standard through the procedure as described in method b).

Example 3: Detection of 2-AP in Different Parts of the Lettuce

Different parts of lettuce (heart stem, leaves, roots, and the milk secreted upon wounding of the peel, and the peels (milk included)) were separated from a single lettuce plant and the weight for each part is listed in Table 1. Each part was analyzed with SPME-GC/MS method. Except for the milk (0.1 g), all the other parts were analyzed with 2 gram of sample. The result is shown in Table 1. Heart stem which is heaviest part of the lettuce shows highest level of 2-AP.

TABLE 1
Weight of different parts from a single lettuce plant and
their respective 2-AP levels.

| | | | Peak area in SPME- |
|---|---|---|---|
| | Total | Sample | GC-MS analysis |
| | weight | weight | (defining the 2-AP |
| Part | (g) | (g) | levels) |
| milk | <5 | 0.1 | 1793528 |
| peel (milk included) | 100 | 2 | 2401634 |
| heart stem | 390 | 2 | 3195112 |
| leaves | 55 | 2 | 2601934 |
| root | 5 | 2 | 385238 |

Example 4: 2-AP Increases Significantly in Cooked Lettuce

1 g of powdered lettuce heart stem (−80° C.) was put in a 20 mL headspace sampling vial. Several vials were prepared like this and they were incubated at different temperatures (25, 45, 70, 100° C.) for 30 min, respectively. After the incubation, all the samples were allowed to cool to room temperature and then analyzed with the SPME-GC/MS (FIG. 4). The level of 2-AP increased significantly with the temperature. A temperature of 100° C. was not exceeded, as it is the limit for common cooking condition.

Example 5: Optimization of the Duration for the Incubation

The duration for the incubation was further optimized for 100 C. 1 g of powdered lettuce heart stem (−80° C.) was put in a 20 mL headspace sampling vial. Several vials were prepared like this and they were incubated at 100° C. for different durations (10, 20, 30, 40, 50 min) respectively. The samples were allowed to cool to room temperature and then analyzed with the SPME-GC/MS (FIG. 5). Obviously, 40-50 min is the optimal duration for incubation at 100° C. to generate highest level of 2-AP.

### Example 6: Thermal Generation of 2-AP in Different Parts and Different Batches of the Lettuce

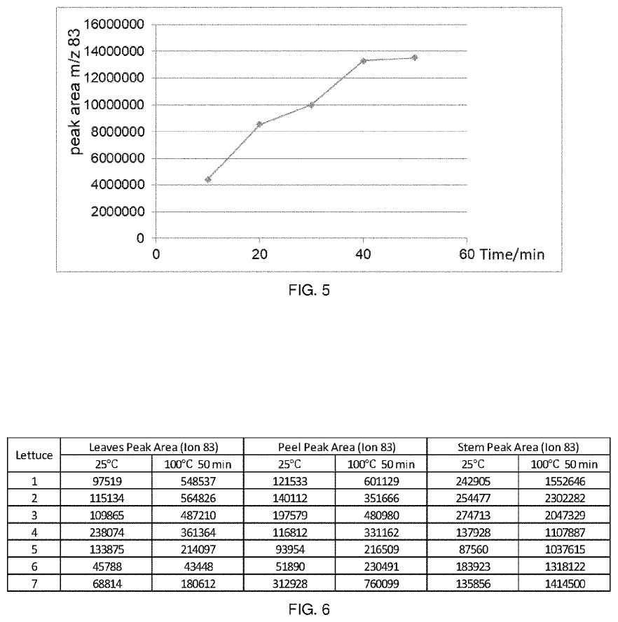

Seven different batches of lettuce were analyzed by parts (leaves, peel, and core stem) for 2-AP with SPME-GC/MS before and after incubation at 100° C. for 50 min. The results are depicted in FIG. 6.

### Example 7: Preparation of Essence from Cooked Lettuce

40 g powdered heart stem of the lettuce was placed in a glass vial and sealed tightly and then incubated at 100° C. for 1 h. After the sample was cooled to room temperature, it was transferred to a flask for distillation. The distillation was carried out with a Solvent Assisted Flavor Extraction apparatus (SAFE) at 65° C. and vac uum was adjusted to keep the sample slightly boiled. The distillation was finished in 0.5 hour and the distillate as the aroma water of lettuce stem was collected. The aroma water smelled typical like the lettuce stem (green +aroma rice). Further SPME-GC/MS analysis of the aroma water showed that the green odor mainly comes from C₆ alcohols and aldehydes.

### Example 8: Preparation of Basic Fraction of the Essence

To remove the green note from the essence, the essence of Example 7 was further processed by acid/base extraction.

The aroma water was first acidified with 10% sulfuric acid to pH=3 and then washed with pentane for three times to remove neutral molecules. The acidic water was recovered and then neutralized with Na₂CO₃ to pH 8. SPME-GC/MS analysis of this basic fraction showed mainly 2-AP in its volatile profile.

Example 9: Stability of 2-AP in the Basic Fraction of the Essence

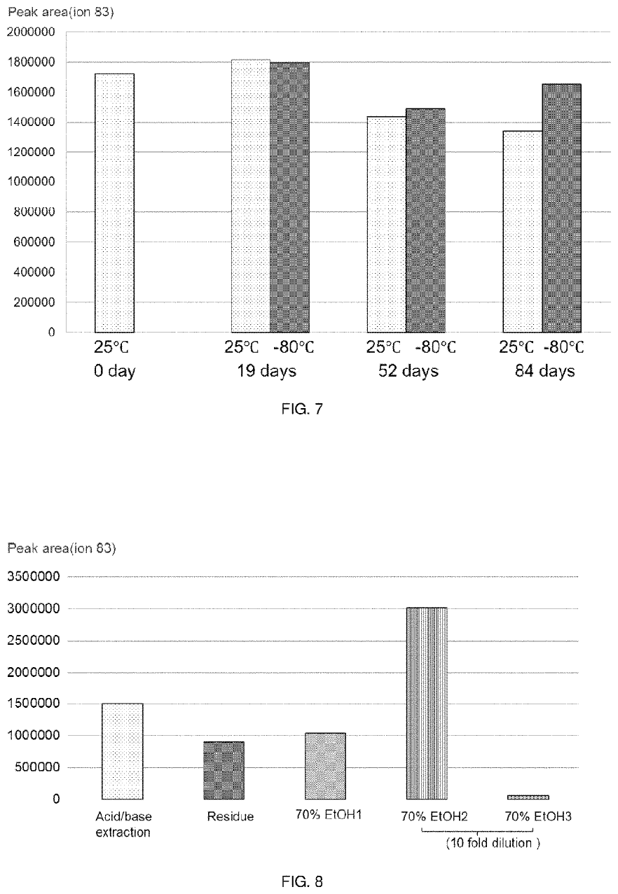

The basic fraction was closed tightly and put at room temperature. A control sample was frozen at −80° C. The samples at room temperature a nd −80° C. were analyzed at different time points (0, 19, 52, and 84 d). Minor degradation of 2-AP was observed on day 52 and notable degradation was observed on day 84. (FIG. 7)

### Example 10: Preparation of Concentrated Essences by Solid Phase Extraction (SPE)

The lettuce essence of Example 8 was further concentrated by SPE. 200 g of basic fraction of the lettuce essence prepared according to the procedure described in Example 8 was loaded through a C18 column (1 g). The column was then eluted with 70% ethanol for three times, each for 1 mL. Each of the fractions (100 uL) was diluted for 10 times to 1 mL with water and analyzed with SPME-GC/MS. Fraction 2 showed the highest level of 2-AP for about 15 fold concentration of the original essence (FIG. 8). Both Fraction 1 and Fraction 2 smelt strong and clean rice aroma.

### Example 11: Precursor Studies

For showing the effect of the precursors of 2-AP for 2-AP formation during a thermal process, the slurry of lettuce heart stem (powdered sample warmed to room temperature) was centrifuged to separate the supernatant and the residue. The residue was further washed with water for three times. Four samples were prepared in 20 mL headspace sampling vials as:

Sample 1: 1 g supernatant;

Sample 2: 0.5 g supernatant+0.5 g residue;

Sample 3: 0.5 g supernatant+0.5 g water,

Sample 4: 1 g residue.

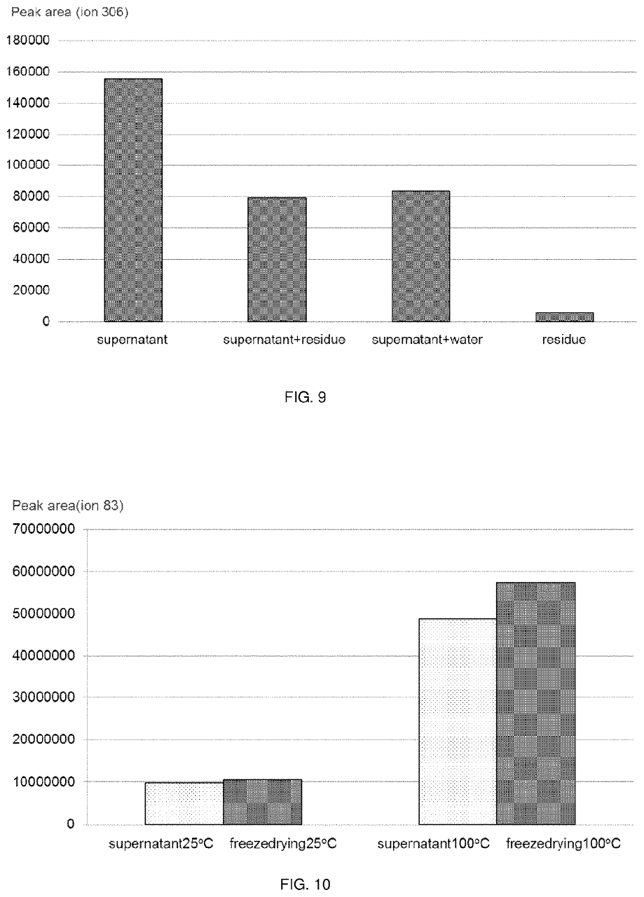

All the samples were incubated at 1001 for 50 min and the content of 2-AP was determined by the derivatization method (FIG. 9).

Sample 1 showed twice as much as the level of 2-AP in Sample 2 and 3, whereas in Sample 4 the level of 2-AP was very low, which show that all necessary precursors for 2-AP generation in the thermal process exist in the supernatant and that the residue contains only little amounts of precursors.

10 g supernatant was further lyophilized for 2 days to remove water and volatiles as much as possible. The residue after lyophilization was reconstituted with water to 10 gram. The reconstituted supernatant and normal supernatant was analyzed for 2-AP with SPME-GC/MS method before and after incubation at 100° C. for 50 min.

The analyses showed that the reconstituted supernatant showed even a little more 2-AP than the normal supernatant for both before and after the incubation (FIG. 10), which indicated that all the necessary precursors for 2-AP generation in heat process are less volatile components.

### Example 12: Rice Cooked with Lyophilized aqueous Lettuce Extract

To test the proflavor property of lyophilized aqueous lettuce extract, 60 g core stem supernatant was lyophilized for 24 h, and 3.2 g of light green powder was obtained. 100 g of normal rice was cooked in a rice cooker together with 1.6 g of the lyophilized powder. A control sample was cooked without adding the lettuce extract. Both samples were cooled to room temperature and evaluated by 12 panellists (chewing and swallowing), 11 of them tasted clearly much stronger rice aroma and 3 of them thought the sample cooked with the lettuce extract tasted sweeter.

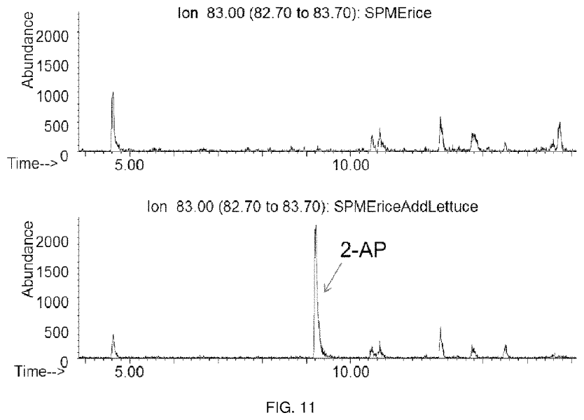

The two samples (2 g) were also analyzed with SPME-GC/MS. 2-AP was observed in the sample cooked with the lettuce extract but not in the control sample (FIG. 11).

### Example 13: Lettuce Powder 20.3kg of fresh lettuce was subjected to the following operations:

### Juicing

The fresh lettuce was squeezed in a juicer for 2 times to obtain 14.4 kg of juice.

### Filtration

The juice was fed into a microfiltration plant with the membrane size of 0.1 μm. 8.7 kg of filtrate was obtained after filtration.

### Spray drying

The carrier, 0.06 kg Capsul and 0.56 kg Maltodextrin18 DE, was added to the filtrate and mixing for 30 min. The solution was introduced into spray dry with the flowrate of 400 ml/hr. 0.6 kg of product was obtained.

1. An extract from lettuce (Lactuca sativa) comprising 2-acetyl-1-pyrroline and, optionally, precursors of 2-acetyl-1-pyrroline.

2. The extract according to claim 1, wherein the lettuce (Lactuca sativa) is stalk lettuce (Lactuca sativa var. angustana).

3. The extract according to claim 1, wherein the extract is obtained from the leaves, peels, roots or the core stem of the lettuce or any mixture thereof.

4. The extract according to claim 1, wherein the extract is an aqueous extract or a powdered extract.

5. A flavoring composition comprising:
i. at least an extract from lettuce (Lactuca sativa), preferably stalk lettuce (Lactuca sativa var. angustana), comprising 2-acetyl-1-pyrroline and, optionally, precursors of 2-acetyl-1-pyrroline;
ii. at least one ingredient selected from the group consisting of a flavor carrier, a flavoring co-ingredient and a mixture thereof; and
iii. optionally, at least one flavor adjuvant.

6. A flavored consumer product comprising an extract according to claim 1.

7. A method of using an extract from lettuce (Lactuca sativa) comprising 2-acetyl-1-pyrroline and, optionally, precursors of 2-acetyl-1-pyrroline, the method comprising using the extract as a flavoring ingredient.

8. A method to confer, enhance, improve, or modify the taste properties of a flavoring composition or of a flavored article, wherein the method comprises adding to said composition or article an effective amount of an extract from lettuce (Lactuca sativa), comprising 2-acetyl-1-pyrroline and, optionally, precursors of 2-acetyl-1-pyrroline.

9. A method for the preparation of an extract of lettuce (Lactuca sativa) comprising 2-acetyl-1-pyrroline and, optionally, precursors of 2-acetyl-1-pyrroline, the method comprising the following steps:
a) providing the lettuce (Lactuca sativa), and
b) isolating therefrom an extract comprising 2-acetyl-1-pyrroline and, optionally, precursors of 2-acetyl-1-pyrroline.

10. The method according to claim 9, wherein step b) comprises the following steps:
b.1.) thermally processing the lettuce to obtain a processed lettuce,
b.2.) subjecting the processed lettuce of step b.1.) to hydrodistillation or vacuum distillation, and
b.3.) obtaining the extract as the distillate.

11. The method according to claim 10, wherein step b.1.) occurs at a temperature selected from the range of 25° C. to 100° C.

12. The method according to claim 10, wherein step b.1.) lasts for a period of time selected from the range of 1 to 50 min.

13. The method according to claim 10, wherein the method comprises the following additional step(s)
b.4.) subjecting the extract obtained in step b.3. to acid/base extraction,
optionally, b.5.) subjecting the extract obtained in step b.4.) to a further concentration step.

14. The method according to claim 9, wherein step b) comprises the following steps:
b.1.) dissolving the lettuce in an aqueous solution to obtain a lettuce residue and a supernatant;
b.2.) obtaining the extract as the supernatant.

15. The method according to claim 9, wherein the method comprises the following step:
c) drying the extract.

16. The extract according to claim 3, wherein the extract is obtained from the leaves, peels or the core stem of the lettuce or any mixture thereof.

17. A flavored consumer product comprising the composition according to claim 5.
